(19) United States
(12) Patent Application Publication
(10) Pub. No.: US 2002/0193308 A1
(43) Pub. Date: Dec. 19, 2002

(54) Recombinant protein and its use in therapy and diagnostics

(75) Inventors: Vytautas Naktinis, Milano (IT); Daniela Concas Benevelli, Milano (IT); Bruno Berra, Milano (IT); Irma Colombo, Milano (IT); Severino Ronchi, Milano (IT); Alberto Bartorelli, Milano (IT); Viadas Algirdus Bumelis, Milano (IT)

Correspondence Address: BIRCH STEWART KOLASCH & BIRCH, PO BOX 747, FALLS CHURCH, VA, 22040-0747, US

(73) Assignee: ZETESIS S.P.A.

(21) Appl. No.: 10/147,454

(22) Filed: May 17, 2002

Related U.S. Application Data
(63) Continuation of application No. 09/291,332, filed on Apr. 15, 1999.

(51) Int. Cl. A61K 38/17; C07H 21/04; C07K 14/435; C12P 21/02; C12N 5/06

(52) U.S. Cl. 514/12; 530/350; 536/23.5; 435/69.1; 435/320.1; 435/325

(57) ABSTRACT

A new protein obtained through recombinant DNA techniques which can be used in diagnostics and in therapy, in particular for treating tumors, a cDNA molecule encoding such a protein, a process for preparing it, and expression vectors and host cells which may be used in such a process.

### CROSS-REFERENCE TO RELATED APPLICATION

This is a continuation of U.S. patent application Ser. No. 09/291,332, which was filed on Apr. 15, 1999. The entire contents of application Ser. No. 09/291,332 are hereby expressly incorporated by reference. Priority under 35 USC 120 is claimed to the Apr. 15, 1999 filing date of application Ser. No. 09/291,332.

### FIELD OF THE INVENTION

The present invention relates, in general, to a protein of therapeutic interest. In particular, the invention concerns a new recombinant UK114 protein, its cDNA, and the use of this protein in therapy and diagnostics.

### BACKGROUND OF THE INVENTION

WO 92/10197 discloses extracts of mammalian organs, particularly of goat liver (UK 101), consisting of at least three different proteins and characterized by unusual pharmacological and immunological properties, which suggested their use as anticancer agents.

WO 96/02567 discloses a protein purified from the extract disclosed in WO 92/10197. This protein, which is named UK114, has a molecular weight of about 14 kDa, has a marked antineoplastic activity, and is capable of raising, in animals including humans, antibodies which recognize human carcinoma cells. The amino acid sequence (SEQ ID NO:3) of UK114 has recently been determined by automated amino acid sequencing. See Colombo, et al., Biochimica et Biophysica Acta, 1442: 49-59, (1998).

Due to the very interesting pharmacological properties of UK114, many efforts have been directed towards the preparation of a recombinant UK114 protein. The preparation of a recombinant UK114 molecule has recently been reported by Irma Colombo, et al., “cDNA cloning and Eschenchia coli expression of UK114 tumor antigen”, Biochimica et Biophysica Acta, 1442: 49-59, (1998). The sequence of this recombinant protein has been established to be that reported herein in SEQ ID NO:2.

From comparing the sequence of SEQ ID NO:2 (a prior art recombinant UK114) with that of the UK114 protein extracted from goat liver (SEQ ID NO:3), it appears that the former has two extra amino acid residues (Val-Pro) in the amino-terminal region. This is a consequence of a cloning-artifact due to the use of the particular expression vector pTrxFus.

Colombo, et al. report a strong immunoreactivity of this recombinant protein to rabbit antisera prepared against UK101 or against UK114 purified from goat's liver and to sera of UK101-treated cancer patients. This indicate that the presence of two extra amino acid residues in the NH₂-terminus of the recombinant UK114 does not alter its biological activity.

Nevertheless, there still exists a strong need for a recombinant UK114 which is as close as possible to natural UK114.

### SUMMARY OF THE INVENTION

An object of the present invention is therefore that of providing a recombinant UK114 protein which shows the closest possible similarity to natural UK114. This object has been achieved by the provision of the recombinant UK114 protein having the sequence of SEQ ID NO: 1.

By comparing the sequence of SEQ ID NO:3 and that of the recombinant protein according to the present invention (SEQ ID NO: 1), it appears that the latter differs from the natural UK114 only in the first amino acid (SER)of the amino terminus, the amino acid terminus itself not being acetylated.

The invention also provide a cDNA molecule, encoding the new recombinant UK114 protein, having a nucleotide sequence in accordance with SEQ ID NO:4, and an expression vector comprising such nucleotide sequence. In addition, the present invention provides a prokaryotic or eukaryotic host cell transformed with the above-mentioned expression vector and also a process for preparing the recombinant protein, which comprises the following steps:

constructing DNA having a nucleotide sequence in accordance with SEQ ID NO:4 encoding the desired protein;

inserting said DNA into an expression vector;

transforming a host cell with this recombinant DNA (rDNA);

culturing the transformed host cell to express the recombinant protein;

extracting and purifying the recombinant protein so produced.

The novel recombinant protein of the present invention can be used in anti-tumor therapy and in diagnostics.

### DETAILED DESCRIPTION OF THE INVENTION

In one embodiment, the present invention provides a purified protein having the amino acid sequence of SEQ ID NO: 1. As discussed above, this is a recombinant UK114 protein. The present invention also provides a method of treating tumors in human beings comprising administering to a human being in need of such treatment a therapeutically effective amount of that protein, and a pharmaceutical composition containing as its active substance that protein in admixture with a pharmaceutically acceptable carrier.

Another aspect of the present invention contemplates a purified recombinant protein having the amino acid sequence of SEQ ID NO: 1, which has been obtained by constructing a DNA encoding said sequence; inserting said DNA into an expression vector; transforming a host cell with the vector containing the recombinant DNA; culturing the transformed host cell so as to express the recombinant protein; and extracting and purifying the recombinant protein so produced. The present invention also contemplates that synthetic procedure, and a method of treating tumors in human beings comprising administering to a human being in need of such treatment a therapeutically effective amount of the protein so produced, as well as a pharmaceutical composition containing as its active substance that protein in admixture with a pharmaceutically acceptable carrier.

Finally, this invention includes an isolated cDNA molecule encoding the protein described above, the cDNA molecule having a nucleotide sequence in accordance with SEQ ID NO:4, as well as an expression vector comprising the nucleotide sequence according to SEQ ID NO:4, and a prokaryotic or eukaryotic host cell transformed with that expression vector.

The recombinant UK114 according to the present invention has been obtained as illustrated in the following non-limiting examples.

### EXAMPLE 1

### Recombinant Gene

This example relates to preparation of a manufactured gene encodingUK114 and including E.coli preference codons. Briefly stated; the procedure employed included, first, examination of the 5′-end of the coding region of the UK114 cDNA sequence for the presence of rare codons for E.coli, encoding for the arginine amino acid residue. As illustrated in Table I, one such codon AGA was identified at position Arg5. Using the degeneracy of the genetic code, this codon could be changed into CGT without causing substitution of the amino acid residue.

TABLE I

| | |
|---|---|
| Original | ATG TCG TCT TTG GTC AGA AGG (SEQ ID NO:5) |
| sequence | Met0 Ser1 Ser2 Leu3 Val4 (SEQ ID NO:6) |
| | Arg5 Arg6 |
| Modified | ATG TCG TCT TTG GTC CGT AGG (SEQ ID NO:7) |
| sequence | Met0 Ser1 Ser2 Leu3 Val4 (SEQ ID NO:6) |
| | Arg5 Arg6 |

The change indicated was carried out using the pTrx-Fus-UK114 plasmid (Colombo, et al.) and the oligonucleotide primers as indicated in Table II. The use of the primers allowed the simultaneous insertion of the NdeI and HindIII restriction endonucleases cleavage sites at the ends of the DNA fragment generated. The protruding 3′-dA ends were characteristic of the DNA fragment produced.

TABLE II

| Primer | Sequence 5′→ 3′ |
|---|---|
| UK-1 | CATATGTCGTCTTTGGTCCGTAGG (SEQ ID NO:8) |
| (modified 5′-terminus of | ATAATCAGC |
| UK114) | |
| UK-2 | AAGCTTTTAGAGTGATGCTGTCGT (SEQ ID NO:9) |
| (3′-terminus of UK114) | |

The 423 bp PCR-product generated in a 25-cycle PCR (1 min steps at 94° C., 50° C., and 72° C.) was purified from the reaction components by agarose gel electrophoresis. This DNA fragment was ligated into pUC57/T vector (MBI Fermentas, Vilnius, Lithuania), which is suitable for the effective insertion of DNA fragments with 3′-dA protruding ends. The ligation product was then used to transform an E.coli strain suitable for screening for the correct plasmid structure (e.g. E. coli XL 1-Blue). The plasmid from the selected clone was then digested with NdeI restriction endonuclease, and then blunt-ended with Poll (Klenow fragment). Next, the DNA generated was digested with HindIII restriction endonuclease so as to produce a 420 bp DNA fragment, which was subsequently purified by agarose gel electrophoresis. This DNA fragment constituted the manufactured gene of the UK114 in accordance with the present invention (SEQ ID NO:4).

### EXAMPLE 2

### Recombinant Vector

This example is related to a procedure for construction of an E.coli transformation vector incorporating UK114 encoding DNA. The vector is used in the prokaryotic expression of UK114.

Although any suitable vector may be employed to express the manufactured gene of UK114, the expression plasmid pKK-UK114 may readily be constructed from a plasmid pKK223-3, the structure of which is described in Amersham Pharmacia Biotech catalogue BioDirectory '98, cat No. 27-4935-01. Plasmid pKK223-3 is first cleaved with a mixture of BamHI and Eco471II restriction endonucleases, in order to delete a 1352-bp fragment, which contains a tetracycline resistance gene (position 375 to 1727 according to the pBR322 numbering system). The remaining DNA fragment is blunt-ended with Poll (Klenow fragment), circularized by ligation through the blunt ends, transformed into a suitable E.coli strain (e.g. E.coli K12 JM105, cat No. 27-1550-01, Amersham Pharmacia Biotech), and the resulting plasmid vector designated as pKK223-3ΔTc is purified from selected clone.

Then, the pKK223-3ΔTc vector is cleaved with EcoRI restriction endonuclease and blunt-ended with Poll (Klenow fragment). Next, the obtained linearized plasmid DNA is digested with HindIII restriction endonuclease, and ligated with a 420 bp manufactured gene of UK114. The E.coli JM 109 cells are transformed with this ligation product, to give the expression vector pKK-UK114 in the host strain of E.coli JM 109. The product of this procedure is an expression plasmid containing a continuous DNA sequence, as shown in SEQ ID NO:4, encoding the entire UK114 polypeptide with an amino terminal methionine [Met0] codon ATG for E.coli translation initiation. Control of expression in the expression pKK-UK114 vector is by means of a tac promoter, which is inducible by isopropyl-β-D-thiogalactopyranoside (IPTG).

### EXAMPLE 3

### Expression and Purification of Recombinant UK114

This example relates to an E.coli expression of an UK114 polypeptide by means of a DNA sequence encoding UK114, and development of purification procedures of recombinant UK114. The sequence employed for expression was partially synthetic and partially cDNA-derived. The synthetic sequence employed E.coli preference codons.

Culture of cells in LB broth (ampicillin 50 μg/ml) was maintained at 37° C., and upon growth of cells in culture to optical density of ˜1 at the UK114 expression was induced by addition of IPTG to the final concentration of 1 mM. Cultivation was continued for 3 more hours at 37° C. The final optical density of the culture was ˜2.5.

The level of expression of UK114 by the transformed cells was estimated on a SDS-containing polyacrylamide gel (SDS-PAGE) stained with coommassie blue dye to be 3-5% of total cellular protein.

Cells from 500 ml fermentation broth were harvested by centrifugation at 3,500 xg for 20 min, re-suspended in 1/50 volume of 10 mM Tris-HCl containing 1 mM EDTA (pH 8.0), and subjected to ultrasonication for 2 min. Cell homogenates were clarified by centrifugation at 40,000 xg for 20 min, and supernatants were applied on a TSK-G2000SW 21.5 mm×60 cm gel filtration column (LKB, Sweden), equilibrated in the buffer as above. The peak fractions containing UK114 protein as judged by SDS-PAGE were pooled, adjusted to 0.1% TFA final concentration, and subjected to reverse phase HPLC on a Hi-Pore RP-304 (C4) 250 mm×10 mm column (Bio-Rad) in a mobile phase consisting of a gradient of 0-90% acetonitrile in 0.1% TFA at a flow rate of 1 ml/min. The dominant UV-absorbing peak fractions containing UK114 protein were pooled and freeze-dried. As a result of purification, UK114 protein was isolated (-2.5 mg) at a purity of about 90%, as judged by SDS-PAGE.

A second purification procedure was developed to yield larger quantities of UK114 formulated in a stabilised solution suitable for in vivo studies. Fifty grams of cell paste was re-suspended in about 500 ml of 10 mM Tris-HCl (pH 7.5), containing 5 mM EDTA and 2 mM phenylmethylsulfonyl fluoride and passed 3 times through a Manton Gaulin extrusion homogenizer at about 7,000 psi. Ethylene imine polymer (molecular weight 600,000-1,000,000) was added to the cell homogenate to the final concentration in the range of 0.15-0.45%. The mixture was incubated for about one hour, and then the suspension was clarified by centrifugation at 40,000 xg for 20 min. The clarified supernatant was adjusted to pH 7.4 with 0.5 M HCl and diluted to about 3.5 liters, and then applied on a 80 ml Q-Sepharose FF column equilibrated in 10 mM Tris-HCl (pH 7.4). After loading, the column was washed with two column volumes of equilibration buffer. The flow-through material containing UK114 was concentrated to a final volume not greater than 65 ml, by ultrafiltration using a 10 kDa cut-off membrane cassette of 0.75 sq.feet (Filtron, Omega series Minisette OS-01OC-01). The concentrated UK114 containing material was applied to a 350 ml Sephadex G-25 Super Fine column, equilibrated in phosphate buffered saline (0.14 M NaCl, 2 mM KCl, 8 mM sodium phosphate, 1.5 mM potassium phosphate, pH 7.5) containing 0.004% Tween 80. The column was eluted with equilibration buffer, and the fractions of the main protein elution peak comprising about 115 ml were pooled and filter sterilised. The final concentration of UK114 was about 1 mg/ml, the purity of the protein was greater than 95% as determined by SDS-PAGE, and the final formulation was pyrogen-free as determined by European Pharmacopoeia rabbit pyrogenicity test with a test-dose of 100 μg protein in 1 ml water i/v per 1 kg rabbit weight.

### EXAMPLE 4

### Physical Properties of the Polypeptide

This example relates to physical properties of the recombinant polypeptide product of the invention.

Apparent Molecular Mass by Sds-Page.

Recombinant UK114 product of E.coli expression as in Example 3 had an apparent molecular mass of ˜12.1 kDa indistinguishable from that of the natural isolate purified UK114 when determined in SH-reducing SDS-PAGE in a tricine-SDS system (Schagger, H., and von Jagow, G. Anal. Biochem. 166, 368-379 (1987)). This value is different from that expected from the deduced amino acid sequence in SEQ ID NO:3 (i.e. ˜14.2 kDa). This is a reflection of a well established fact, that proteins with molecular mass of ≦14 kDa tend to deviate detectably from the linear relationship of log(molecular mass) vs relative mobility. Consistent with such anomaly, the apparent molecular mass of recombinant UK114 varied in a range from 14.3 to 14.9 kDa (dependent on % of polyacrylamide), when analysed in a Laemmly system of the SDS-PAGE (Laemmli, U. K., Favre, M. J.Mol.Biol., 80, 575-599 (1973)). For these reasons, the use of the values of apparent molecular mass determined for UK114 should be limited to comparative identification analysis, rather than being extended to the characterization of the molecular structure of this protein.

Aggregation State.

The recombinant UK114 product of E.coli expression as in Example 3 exhibited a gel-filtration elution time characteristic of a molecular mass of ˜30 kDa, a value consistent with the dimeric aggregation state of this protein. In this aspect recombinant product was indistinguishable from the natural analogue, as determined by the co-elution of the two proteins in a gel-filtration on a HPLC TSK 2000 7.5 mm×600 mm column at a monomer concentration of ˜28 μM in 10 mM potassium phosphate buffer (pH 7.0) containing 0.3 M NaCl. The two monomers are held in a dimeric state by a non-covalent bonds, as demonstrated by the dissociation of the dimer in 6M guanidinium chloride solution.

N-Terminal and C-Terminal Amino Acid Sequences.

N- and C-terminal sequence analyses were carried out on the recombinant UK114 product of E.coli expression as in Example 3, to demonstrate that the gene is expressed correctly from start to finish and that both ends are not altered in bacterial cells or during the manufacturing process. Using a manual format of Edman degradation sequencing film method with polybrene, performed essentially as described in Tarr, Manual Edman sequencing system. In: Methods of protein micro-characterization (Shively, et al., eds., Humana Press, Clifton, N.J., 1986, 155-194), the phenylthiohydantoin (PTH) derivatives of amino acids were separated and identified by reverse phase HPLC on Nova-Pak C18 column (Waters). Table III shows the quantitative data obtained from the analysis.

TABLE III

| | | Experimental data | |
|---|---|---|---|
| Edman | | | Yield of |
| degradation | Amino acid | PTH-amino | amino acid, |
| cycle | deduced from | acid | nmol/nmol of |
| No | gene sequence | identified | protein |
| 1 | Ser | Ser | 0.87 |
| 2 | Ser | Ser | 0.78 |
| 3 | Leu | Leu | 0.57 |
| 4 | Val | Val | 0.50 |
| 5 | Mg | Mg | 0.43 |
| 6 | Mg | Mg | 0.61 |
| 7 | Ile | Ile | 0.45 |

The results of N-terminal analysis demonstrated that the recombinant UK114 contained only one PTH-amino acid residue after each Edman degradation cycle. This indicates that the protein is homogeneous from its N-terminal end and contained only Ser as the first amino acid at the N-terminus (within the pre-set limit of detection≧5%). The observation that no additional N-terminal acid was found implied that the initiating methionine residue is quantitatively removed as a result of the intracellular methionine aminopeptidase activity. Furthermore, the susceptibility of the protein to Edman degradation indicates that there was no N-terminal block (e.g., acetylation) within the recombinant UK114. Further sequencing data also indicated that the first seven amino acid residues of purified recombinant UK114 (SEQ ID NO: 1) were Ser 1-Ser2-Leu3-Val4-Arg5-Arg6-Ile7- . . . , and as such, the data demonstrated that the recombinant UK114 product is a non-acetylated analogue of its natural counterpart.

A partial C-amino acid sequence was derived by analysing the kinetics of the step-wise carboxypeptidase Y cleavage of C-terminal amino acid residues as previously described. by Jones, B. N., In: Methods of Protein Microcharacterization (Shively, J. E., ed.), 337-361. Humana Press, Clifton, N.J. (1986)). Digestions of 27.5 nmol of UK114 were performed in 0.06 M sodium acetate buffer at 37° C., the buffer having a pH of 5.5. The digestions were carried out with an enzyme to substrate ratio of 1:50 by weight. Aliquots of the carboxypeptidase Y digestion mixture were withdrawn at selected time points of incubation, and amino acids, which had been cleaved-off over the course of enzyme digestion, were treated with phenylisothiocyanate (PITC), and the reverse-phase HPLC of the phenylthiocarbamyl-derivatives (PTC) of amino acids on a Nova-Pak C18-HPLC column was performed. The chromatograms were monitored at 254 nm, and the quantitative evaluation was performed by integration of the peaks of individual amino acids. The identification of PTC-amino acid peaks was based on the comparison of their retention times with those of the PTC-amino acid reference preparations. The results of this analysis demonstrate, that leucine amino acid was released from the C-terminus of recombinant UK114 polypeptide at a substantial rate, followed by serine and alanine. The fourth identifiable amino acid was threonine. These experimental data imply that the C-terminal amino acid sequence (residues 133-136 of SEQ ID NO:1) is Thr133-Ala134-Ser135-Va1136, and establish that the C-terminus of the recombinant UK114 polypeptide is identical to that of its natural counterpart.

Isoelectric Point.

Recombinant UK114 product of E.coli expression as in Example 3, when subjected to isoelectric focusing within the pH range of pH 3.5-10.0, exhibited a major band with an isoelectric point at approximately pI 7.3, and two or three slightly more acidic minor bands, among which the main two position were at approximately pI 7.1, and 6.8. The total amount of the minor isoforms did not exceed 10% of the overall material.

### EXAMPLE 5

### Biological Properties of the Polypeptide

This example relates to biological properties of the recombinant polypeptide product of the invention.

Inhibition of a Cell-Free Protein Synthesis.

Capacity of recombinant, E.coli-derived material to inhibit protein synthesis in a rabbit reticulocyte lysate system was assayed as described in Oka, T. et al. J.Biol.Chem. 270, 30060-30067 (1995) and Schmiedeknecht, G. et al. Eur.J.Biochem. 242, 339-351 (1996). An “in-house” made rabbit reticulocyte lysate assay system using a src RNA was used to measure an incorporation of [35S]-methionine into de novo synthesised src protein in the presence or absence of recombinant UK114. 1 μM concentration of the E.coli recombinant material was found to effectively inhibit protein synthesis in vitro.

Immunoassay.

The polyclonal antibodies (lot RF29), raised in rabbits against natural isolate of UK114 as described in Bartorelli, A. et al. J.Tumor Marker Oncol. 9, 37-47 (1994), were used in a Western blot analysis to demonstrate that the E.coli-derived recombinant UK114 is strongly immunoreactive to these antibodies. A semi-quantitative comparative evaluation of the apparent association constants for recombinant UK114 and natural UK114 with these antibodies was undertaken by analysis of competition binding curves obtained in enzyme-linked immunoassay (ELISA). The antigen solution, containing 10 μg/ml of natural UK114 protein in a coating buffer (50 mM NaHCO₃, pH 9.5), was pipetted into wells of 96-well ELISA plates, 100 μl per well and incubated overnight at 4° C. 200 μl of blocking solution (0.15 M NaCl, 50 mM Na-K phosphate buffer, pH 7.4, containing 0.5% Tween 80) was added into each well of ELISA plates and incubated at ambient temperature for 1 hr. Series of two-fold dilutions of natural, and recombinant UK114 in blocking buffer were prepared, starting with the concentration of 4 μg/ml. 50 μl of each dilution were pipetted into wells along with 50 μl of diluted (1:10,000) rabbit anti-UK114 antiserum, and incubated at ambient temperature for 2 hours. Negative and positive control wells were included in each plate. Negative control wells were used to evaluate the non-specific binding and contained therefore no rabbit antiserum. Positive control was used to establish the maximal possible binding of rabbit antibodies in the absence of any antigen in the reaction mixture. Then ELISA plates were washed 10 times with blocking solution, and anti-rabbit IgG secondary antibodies, conjugated with horse-radish peroxidase (1:1,000 diluted), were added 100 μl/well and incubated for 1 hr at ambient temperature, to detect rabbit antibodies, bound to the immobilised UK114. ELISA plates then were washed 10 times with blocking solution. 4 mg of orthophenilendiamine were dissolved in 10 ml of substrate buffer (25 mM sodium acetate, pH 5.5), and 30 μl of 19% H₂O₂ were added to prepare the substrate-chromogene solution. This solution was added to each well of the plate (100 μl/well) to visualise the bound horse-radish peroxidase. The enzymatic reaction was carried out in dark for 10-15 min and stopped by adding 50 μl/well of 2 M H2SO4. The ELISA plates were quantitatively scanned at λ492 nm, and the mid-point optical density (A50) for each antigen was calculated. Then the apparent association constants (Ka), defined as reciprocal of the respective antigen protein concentration at the A50 point and expressed in M-1 were calculated. It was determined that the Ka were 1.29×109 M-1, and 0.49×109 M-1, for natural UK114, and recombinant UK114, respectively. This apparent difference m specific antibody binding could be related to the absence of N-terminal acetylation in the E.coli-derived polypeptide when compared to its natural analogue.

What is claimed is:

1. A purified protein having the amino acid sequence of SEQ ID NO:1.

2. A method of treating tumors in human beings comprising administering to a human being in need of such treatment a therapeutically effective amount of the protein of claim 1.

3. A pharmaceutical composition containing as its active substance the protein of claim 1 in admixture with a pharmaceutically acceptable carrier.

4. A purified recombinant protein having the amino acid sequence of SEQ ID NO: 1, which has been obtained by a process comprising the following steps:
a) constructing a DNA encoding said sequence;
b) inserting said DNA into an expression vector;
c) transforming a host cell with the vector containing the recombinant DNA;
d) culturing the transformed host cell so as to express the recombinant protein; and
e) extracting and purifying the recombinant protein so produced.

5. A method of treating tumors in human beings comprising administering to a human being in need of such treatment a therapeutically effective amount of the protein of claim 4.

6. A pharmaceutical composition containing as its active substance the protein of claim 4 in admixture with a pharmaceutically acceptable carrier.

7. An isolated cDNA molecule which encodes the protein of claim 1 and which has a nucleotide sequence in accordance with SEQ ID NO:4.

8. An expression vector comprising the nucleotide sequence according to SEQ ID NO:4.

9. A prokaryotic or eukaryotic host cell transformed with the expression vector of claim 8.

10. A transformed prokaryotic host cell according to claim 9, wherein the host cell is Escherchia coli JM 109.

11. A process for preparing a recombinant protein having the nucleotide sequence of SEQ ID NO: 1, which comprises the following steps:
a) constructing a DNA encoding said sequence;
b) inserting said DNA into an expression vector;
c) transforming a host cell with the vector containing the recombinant DNA;
d) culturing the transformed host cell so as to express the recombinant protein; and
e) extracting and purifying the recombinant protein so produced.


SEQUENCE LISTING (9 sequences)

SEQ ID NO: 1 - protein, 136 aa, Capra hircus
SSLVRRIISTAKAPAAIGPYSQAVLVDRTIYISGQLGMDPASGQLVPGGVVEEAKQALTNIGEILKAAGCDFTNVVKATVLLADINDFSAVNDVYKQYFQSSFPARAAYQVAALPKGGRVEIEAIAVQGPLTTASV

SEQ ID NO: 2 - protein, 139 aa, Capra hircus
VPMSSLVRRIISTAKAPAAIGPYSQAVLVDRTIYISGQLGMDPASGQLVPGGVVEEAKQALTNIGEILKAAGCDFTNVVKATVLLADINDFSAVNDVYKQYFQSSFPARAAYQVAALPKGGRVEIEAIAVQGPLTTASV

SEQ ID NO: 3 - protein, 137 aa, Capra hircus
MSSLVRRIISTAKAPAAIGPYSQAVLVDRTIYISGQLGMDPASGQLVPGGVVEEAKQALTNIGEILKAAGCDFTNVVKATVLLADINDFSAVNDVYKQYFQSSFPARAAYQVAALPKGGRVEIEAIAVQGPLTTASV

SEQ ID NO: 4 - DNA, 423 bp, Capra hircus
catatgtcgtctttggtccgtaggataatcagcacggcgaaagcccccgcggccattggtccctacagtcaggctgtgttagtcgacaggaccatttacatttcaggacagctaggtatggaccctgcaagtggacagcttgtgccaggaggggtggtagaagaggctaaacaggctcttacaaacataggtgaaattctgaaagcagcaggctgtgacttcacgaatgtggtaaaagcaacggttttgctggctgacataaatgacttcagtgctgtcaatgatgtctacaaacaatatttccagagtagttttccggcgagagctgcttaccaggttgctgctttgcccaaaggaggccgtgttgagatcgaagcaatagctgtgcaaggacctctcacgacagcatcactctaaaagctt

SEQ ID NO: 5 - DNA, 21 bp, Artificial Sequence
  5′ end of the coding region of the UK114 cDNA sequence derived from Capra hircus
atgtcgtctttggtcagaagg

SEQ ID NO: 6 - protein, 7 aa, Artificial Sequence
  5′ end of the amino acid sequence derived from the coding region of the UK114 cDNA sequence derived from Capra hircus
MSSLVRR

SEQ ID NO: 7 - DNA, 21 bp, Artificial Sequence
  Modified 5′ end of the coding region of the UK114 cDNA sequence derived from Capra hircus
atgtcgtctttggtccgtagg

SEQ ID NO: 8 - DNA, 33 bp, Artificial Sequence
  UK-1 Primer modified 5+40-terminus of UK114 derived from Capra hircus
catatgtcgtctttggtccgtaggataatcagc

SEQ ID NO: 9 - DNA, 24 bp, Artificial Sequence
  UK-2 Primer  3′-terminus of UK114 derived from Capra hircus
aagcttttagagtgatgctgtcgt